(19) United States
(12) Patent Application Publication
(10) Pub. No.: US 2004/0191242 A1
(43) Pub. Date: Sep. 30, 2004

(54) Method for producing a vaccine

(76) Inventors: Gottried Himmler, Vienna (AT); Hans Loibner, Vienna (AT); Helmut Eckert, Obervil (CH); Otto Doblhoff-Dier, Baden (AT); Ralf Kircheis, Vienna (AT); Manfred Schuster, Schrick (AT); Erich Wasserbauer, Vienna (AT); Gunter Waxenecker, Mank (AT)

Correspondence Address: BIRCH STEWART KOLASCH & BIRCH, PO BOX 747, FALLS CHURCH, VA, 22040-0747, US

(21) Appl. No.: 10/472,876

(22) PCT Filed: Mar. 20, 2002
(86) PCT No.: PCT/AT02/00091

(30) Foreign Application Priority Data
Mar. 28, 2001 (AT) A470/2001

(51) Int. Cl. A61K 39/395

(52) U.S. Cl. 424/130.1

(57) ABSTRACT

There is disclosed a method of producing an autologous-antibody-containing vaccine, which method is characterized by the following steps:

providing an antibody-containing fluid from an autologous-antibody-containing body fluid or from autologous cell or tissue preparations,

treating the antibody-containing fluid with a solid carrier on which ligands have been immobilized which bond to a certain group of antibodies, with the proviso that, as said ligands, no antibodies or their fragments with the same idiotype are used which are directed against tumor-associated antigens,

recovering the antibodies which bond to the ligands, and

working up the recovered antibodies to an autologous vaccine which comprises an efficient amount of a few micrograms to up to one gram of antibodies.

The present invention relates to a method of producing a vaccine.

Higher organisms are characterized by an immune system which protects them from potentially hazardous substances or microorganisms. If a substance (antigen) enters the body, it is recognized as “foreign” and eliminated with the help of the immune system. Also “degenerate” endogenous cells are usually recognized by the immune system and removed.

The adaptive immune system of humans consists of two essential components, the humoral and the cellular immunity. The adaptive immune response is based on the clonal selection of B- and T-lymphocytes and in principle allows for the recognition of any desired antigen as well as for the build-up of an immunological memory. These characteristics of the adaptive immune system are generally usefully addressed in vaccinations.

Each B-cell produces an antibody with a defined binding specificity. This antibody is also present as a specific receptor in the membrane of the B-cell producing it. The humoral immune response against antigens recognized as foreign is based on the selective activation of those B-cells which produce such antibodies that can bind to epitopes of the respective antigen. For the antibody diversity, DNA rearrangements in the course of B-cell differentiation play a decisive role.

In human serum, there are large amounts of antibodies of the most varying specificities, isotypes and subclasses. The total concentration of all immunoglobulins in the serum is 15-20 mg/ml; this means that about 100 g of immunoglobulins of the most varying specificities continuously circulate in blood. It is not possible to indicate the precise number of all antibodies with different specificity, the repertory of different B-cell clones in one human being is about 10⁹. In general, a certain antibody can bind various similar antigens, even though with different affinity and avidity.

With the help of endogenous regulating mechanisms, the immune system must maintain a homeostasis as regards the distribution and importance of these different specificities. One essential mechanism for this is the “idiotypic network” (Ann. Immunol. 125C: 373-89 (1974)). Against each idiotype of an antibody which determines the binding specificity of the latter, there exist anti-idiotypic antibodies which therefore bind to the idiotype of the first antibody as in an antigen recognition. According to this explanation model, the interactions between the idiotype-specific receptors on lymphocytes are responsible for the regulation of the immune system. These interactions apparently do in fact occur, since it has been shown that in the course of an immune response, also anti-idiotypic antibodies form against the antibodies primary-induced by the immune response. Since thee exist anti-idiotypic antibodies form against any antibody, lymphocytes basically are not tolerant relative to idiotypes of antibodies.

There are several possible ways of interfering in the immune system.

1. Passive antibody therapy:

For therapeutic purposes, it is possible to supply to an organism antibodies required for a certain function within this organism. This type of application is called passive immunotherapy, and it can be used in various medical indications, e.g. in the immunotherapy of cancer (Immunol. Today (2000), 21:403), intoxications (Toxicon (1998), 36:823; therapie (1994), 49:41) and infections (Clin. Infect. Dis. (1995), 21:150). In these cases, antibodies can be used which either have been derived from appropriately immunized animals or can be recovered from cells by various biological or molecular-biological techniques (e.g. hybridoma technique, phage-display technique, etc.) via the immortalization of immunoglobulin genes.

The passive antibody administration has the disadvantage that it does not have a long-lasting effect, since the effect decreases with the natural degradation of the administered antibodies in the recipient organism.

2. Active immunization:

To modulate the immune system, an immunization with antigens can be used. Antigens are molecules, molecule complexes or whole organisms to which antibodies can bind.

Not all the antigens induce an immune response, i.e. not all the antigens are immunogenic. Certain small molecules are not noticed by the immune system (haptens), such smaller molecules can be presented to the immune system in suitable form, and thus be made immunogenic. Such a method is the coupling of the hapten to an immunogenic molecule, a so-called carrier molecule.

Other non-immunogenic antigens are so-called self-antigens, i.e. structures which are recognized by the immune system as endogenous substances. Immunization with such antigens usually does not lead to a specific immune reaction. In case of tumor-associated antigens, the fact that these antigens actually are self-antigens is one of the greatest difficulties in the development of a potent vaccine.

An active immunization against pathogens, such as viruses or bacteria, by means of complete antigens is only possible if the pathogens are attenuated or killed. With attenuated pathogens there is the risk that a reversion will occur, i.e. that the attenuated pathogens turn into virulent forms again. A solution to such a problem could be the use of anti-idiotypic antibodies as surrogate antigens for immunization purposes (Int. Arch. Allergy Immunol. (1994), 105:211).

Active immunization with anti-idiotypic antibodies has also been suggested for the treatment of allergies (Int. Arch. Allergy Immunol. (1999), 118:119).

However, antibodies against endogenous antigens are present in the serum of every human being and are called “natural auto-antibodies”.

Anti-idiotypic antibodies of such natural auto-antibodies are involved in the regulation of these auto-antibodies (Immunol. Reviews (1989), 110:135; Eur. J. Immunol. (1993), 23:783).

An insufficient idiotypic regulation seems to play a role in a number of autoimmune diseases:

systemic Lupus erythematosus (Autoimmunity (1994), 17:149);

autoimmune thyroiditis (Eur. J. Immunol. (1993), 23:2945);

systemic vasculitis (J. Autoimmunity (1993), 6:221);

Guillain-Barre syndrome (Clin. Immunol. Immunopathol. (1993), 67:192);

anti-Factor VII:C autoimmune disease (Proc. Natl. Acad. Sci., USA (1987), 84:828).

The immunization with idiotypic antibodies which mimic autoantigens has already been carried out in autoimmune diseases (J. Rheumatol. (1999), 26:2602). Also peptides have already been described for inducing anti-idiotypic antibodies (Proc. Natl. Acad. Sci., USA (1993), 90:8747; Immunology (1999), 96:333).

The immunization in autoimmune diseases based on T-cell receptors has also already been suggested (J. Immunol. (1990), 144:2167; U.S. pat. No. 6,090,387; U.S. pat. No. 6,007,815).

Active immunization is also used as a protection against toxic substances (e.g. bacterial toxins). If in this case the toxins are to be used as a vaccine, they must previously be attenuated, or inactivated, respectively. Such an inactivation may, however, also influence the effectiveness of the immune response. Anti-idiotypic antibodies as vaccines which mimic the toxins have been suggested (Int. J. Clin. Lab. Res. (1992) 22:28; Clin. Exp. Immunol. (1992) 89:378; Immunopharmacology (1993) 26:225).

It has also been suggested to use anti-idiotypic antibodies when immunizing for the first time so as to achieve then a higher specific immunization effect with a vaccine which alone would be too weak (Virology (1984) 136:247).

3. Withdrawing antibodies and immune complexes from the blood:

One possible way of removing antibodies from the blood is the use of perfusion systems (U.S. pat. No. 5,122,112), which primarily has been applied in autoimmune diseases (The Lancet 10/20/1979), 824). However, such extra-corporeal immune adsorptions constitute a great burden and a high treatment risk for the patient so that this method is not suitable for a broad clinical therapy.

At present, an active immunization for a modulation may be carried out with certain antigens which may either be too toxic or potentially infectious, yet not immunogenic. A partial solution to this problem is the use of anti-idiotypic antibodies for an immunization. However, it is necessary to produce such antibodies either in a cell culture or in vitro, or to induce them in certain organisms and then administer them to another organism.

Therefore, it is an object of the present invention to overcome disadvantages of the prior art, and to provide treatment materials and methods for a plurality of diseases, utilizing the immune system of a patient. In particular, efficient treatment strategies are to be created for autoimmune diseases, allergies and similar disorders.

The present invention therefore has as its object to provide a method of producing an autologous-antibody-containing vaccine, which method is characterized by the following steps:

providing an antibody-containing fluid from an autologous-antibody-containing body fluid or from autologous cell or tissue preparations,

treating the antibody-containing fluid with a solid carrier on which ligands have been immobilized which bond to a certain group of antibodies, with the proviso that, as said ligands, no antibodies or their fragments with the same idiotype are used which are directed against tumor-associated antigens,

recovering the antibodies which bond to the ligands, and

working up the recovered antibodies to an autologous vaccine which comprises an immunogenic amount of more than one microgram of antibodies.

The thus obtained vaccine can now be administered to the patient in a suitable manner. The inventive method solves the initially described problems in that for inducing antibodies against a target antibody in an organism, it is precisely these target antibodies of the same organism that are used. Therefore, neither culturing of the cells in vitro for producing the antibodies is necessary, nor a production in a foreign donor organism. The target antibodies are, e.g., ab1 antibodies having a specificity for an antigen, optionally for inducing anti-idiotypic antibodies, or ab2 antibodies, i.e. anti-idiotypic antibodies for producing an immune response, optionally directed against the anti-idiotype, i.e. practically against the antigen.

Likewise, it is no longer necessary to obtain the inventively obtained antibodies from a pool or plasma pool (cf. Zouali et al., J. Immunol. 135 (2) (1985), pp. 1091-1096). In contrast to such antibody preparations from pools, in which antibodies from various individuals are collected, with the antibody preparations according to the present invention it is possible to produce vaccines that contain 100% autologous antibodies whose effect in the idiotypic network of the individual to be treated can be entirely specific and thereby more effective.

In this manner, an individual specific modulation of the immune system targeted to a respective desired purpose becomes possible. By shifting the immunological balance by administering an autologous vaccine with an inventively produced vaccine, a selective stimulation of those B-cells is ensured which produce antibodies with a certain specificity. The specificity can be determined by the manner of isolating the antibodies (by the choice of the ligands) from an individual.

Preferably, of course, the antibody-containing body liquids are blood, serum, lymph fluid, cerebrospinal fluid, colostrum, mucosal body fluids, such as vaginal secretion or nasal secretion, malignant effusions, faeces or urine, yet autologous cells or tissue preparations obtained by a biopsy, which, by a plurality of methods known per se, can be worked up to antibody-containing fluids to be used according to the invention may just as well be used. According to the invention, primarily those body fluids which have a particularly high antibody content are used as the starting materials, human serum or plasma, of course, being particularly preferred.

What is decisive for the specificity of the inventively obtained vaccines in influencing the idiotypic network is, of course, in each case the choice of the ligand in the present immunoaffinity purification. To recover specific antibody fractions, specific monoclonal or polyclonal antibodies or antigens may be used. Monoclonal antibodies or derivatives thereof can be produced according to methods known per se, such as, e.g., hybridoma technology, phage display technology or recombination. (Immunology Today, 2000, 21: entire issue No. 8). Yet, also idiotype-independent ligands which are able to bind certain specific subclasses or subtypes of immunoglobulin, such as, e.g., one or more of IgG1, IgG2, IgG3 or IgG4, or IgA, IgG, IgM, IgE or IgD, respectively, may be used. Furthermore, ligands may be chosen which recognize a certain group of antibody fragments or antibody chains, e.g. at least parts of the lambda or kappa chains, Fc or Fab fragments. Likewise suitable ligands may selectively bind not only antibodies, but also corresponding isotypes or paraglobulins.

The method according to the invention may further be combined with an alike method for producing an autologous vaccine, wherein, as said ligands, also antibodies or their fragments of the same idiotype may be used which are directed against tumor-associated antigens and/or antibodies. For the purpose of a simple method, it is preferred in this instance that the respective ligands are utilized as a mixture. More complex methods comprise the consecutive or parallel treatment of body fluids with the different ligands. Thereby, e.g., a certain antibody fraction can be recovered from serum, having a specificity for cellular adhesion proteins and/or Lewis Y carbohydrate structures, or having a specificity for B-cell lymphoma, which then is immediately provided as an autologous vaccine formulation.

Autologous vaccines produced according to the invention which comprise antibodies that occur in connection with the B-cell lymphoma, particularly contain only certain sub-classes or fractions of the IgG-containing serum fraction so as to ensure the precisely targeted immune response.

For the isolation according to the invention, e.g., antibodies or antibody fragments, such as, e.g., Fc or Fab fragments, can be used. Ligands may, however, also be other substances to which the immunoglobulins can bind, e.g. ligands for the chromatographic purification of immunoglobulins, affinity peptides, affinity polypeptides, proteins, such as protein A or protein G, or ionic structures which are, e.g., also used for ion exchange chromatography.

In choosing the suitable, immobilized ligands, usually care is taken that an undesired leakage of the ligands or of ligand-antibody complexes into the isolated antibody fraction obtained is avoided. In some instances, also a serial purification of the antibodies may be advisable so as to separate contaminations possibly present by the immobilized ligand. A leakage of the material may, however, also be advantageous if certain ligand-antibody complexes are desired in the preparation.

The production of a vaccine of particularly high quality may comprise the further purification of the recovered antibodies by known methods, such as chromatography, gel permeation, precipitation, separation on a liquid or solid phase, in particular on ferromagnetic particles, or ultra/diafiltration.

In the course of working up, it may be required to prepare the formulation as a stable solution, e.g. by admixing preservatives or complexing substances, or adjuvants, respectively. A storage-stable embodiment of the autologous vaccine produced according to the invention is primarily desirable if the patient is to be immunized several times with the same preparation at certain time intervals.

On the other hand, the administration of vaccines freshly prepared in each case may have the advantage that changes of the immune system are taken into consideration in each case, and the occurrence of escape mutants, such as of antibody-producing cells or infectious agents, can largely be avoided. For this, the simple working up of the recovered antibodies to a vaccine is suitable which, at best, should be right on the spot. Thus, e.g., the vaccine produced according to the invention may be applied immediately after blood has been drawn, within one working day, or even while the patient is being treated.

A further embodiment of the method according to the invention relates to the depletion of components of the body liquid which are not desired in the vaccine. Thus, ligands may be chosen which selectively do not bind certain antibodies, but do bind accompanying substances, to then recover the certain antibodies from the unbound fraction.

By individuals, according to the present invention, individual human or animal organisms are to be understood which have body fluids or tissue that contain antibodies. Preferably, of course, the preparation according to the invention is used in vertebrates, particularly preferred in mammals, in particular, humans.

The isolation of the antibodies from animal body fluids that contain antibodies (e.g. human serum) by immunoaffinity purification can be carried out according to methods known to the person skilled in the art (Clin. Chem. (1999), 45:593; J. Chem. Technol. Biotechnol. 48 (1990), 105). Particularly preferred is a solid phase immunoaffinity purification. In doing so, a specific ligand or a mixture of different ligands is immobilized on a solid phase. The solid phase may be a membrane, a gel, a chromatographic material or a similar material to which ligands can be coupled without a substantial loss of the specific binding properties of these ligands (Mol. Biotechnol. (1994) 1:59).

According to the invention, for immediately producing the vaccine, also a ready-to-use kit may be provided. This kit contains the following components:

a) a device with ligands for binding of a certain group of antibodies from an antibody-containing fluid of an individual,

b) an agent for recovering the antibodies which bond to the ligands, and

c) an agent for working up the recovered antibodies to an autologous vaccine.

The device a) in particular is a container, a tool or an automat for manual or automatic actuation. Device a) contains the ligands charged which optionally are immobilized on a solid carrier. Likewise, a buffer may be contained which allows for the adsorption of the antibodies after the body liquid has been taken up into the device under controlled conditions.

The agent b) comprises substances or solutions of substances for washing or purifying, or desorbing, respectively, the antibodies. Among them are washing buffers and/or elution buffers. Besides, in the inventive kit also a formulating agent including a possible adjuvant is provided, unless this is not already contained as b) in the agent for recovering the antibodies.

It has, e.g., been shown that antibodies can be adsorbed on a poorly soluble aluminum compound, whereupon the latter can simply be washed, re-buffered and confectioned to a finished vaccine in one single device, which vaccine already contains the aluminum compound as an adjuvant. In that case, in the kit according to the invention, only one single means is required for recovering the antibodies and working up the vaccine, instead of separate components b) and c). Optionally, additional auxiliary agents, such as washing and buffering substances, may be provided in the kit.

Suitable ligands may be bound to magnetic beads which can be localized or oriented, respectively, or positioned in a simple manner by using a magnet. A certain ligand is, e.g., bound to ferromagnetic particles and provided in a sterile container for receiving the body fluid. If also a magnetic transporting member or rod is arranged in the container, by switching the magnet on and off, e.g. by means of electric pulses for actuating a solenoid, the particles to which the antibodies from the body fluid are bound can be collected on the transporting member. Subsequently, the particles can be separated, preferably while maintaining the magnetic field. During a washing procedure or the antibody desorption, the magnetic field can be removed again. Subsequently, an immobilization of the particles on the magnet is again suitable so as to respectively separate and recover the washing solution and the desorbed antibodies, respectively.

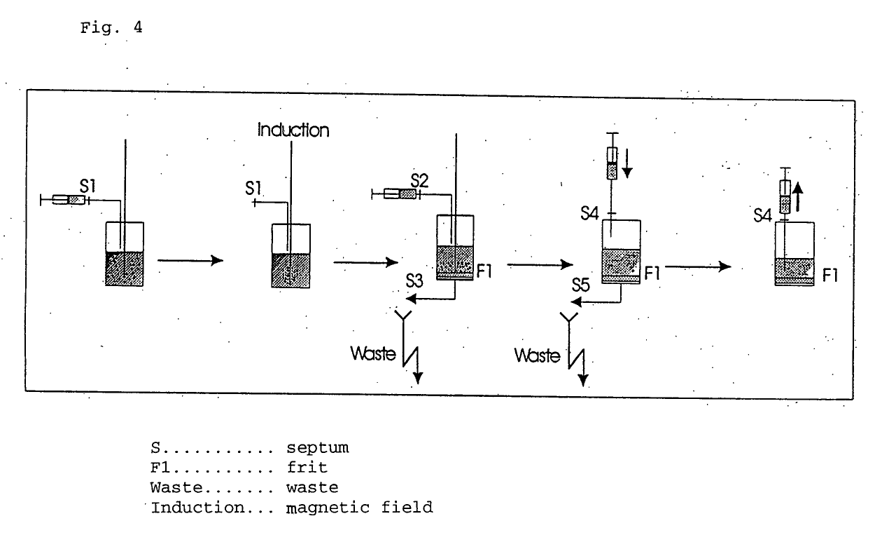

A particular embodiment of a kit for producing an autologous vaccine comprises sterile, endotoxin-free containers, preferably single-use containers which are provided to be used just once, such as syringes which optionally are interconnected and equipped with a septum (cf. FIG. 4 in this context).

For instance, the first container comprises the ligands required for adsorption of the antibodies, bound to ferromagnetic beads. Commercially available beads, are, e.g., activated porous glass beads, such as Prosep (Millipore, Durham, UK), Dynabeads (Deutsche Dynal GmbH, Hamburg, Germany), as well as the same material from Miltenyi (Bergisch Gladbach, Germany). Furthermore, an adsorption buffer is provided in this container, or is charged separately. Via a septum, human serum is introduced. Furthermore, a suitable magnet is provided in or on this vessel so as to immobilize the beads after adsorption, a possible washing, and desorption. Via a septum, a defined amount of washing buffer is introduced. Through a frit, the wash solution is discharged again. Elution of the antibodies can be effected in a separate eluting vessel with elution buffer into which the loaded beads are introduced. After elution of the antibodies, the beads are separated by immobilization on the transporting member and removal of the latter from the solution. Subsequently, a formulating means is added through a septum. The formulated solution is introduced into a syringe and is ready for application on the patient. The individual containers are each equipped with one or more septa for the transfer of sollutions or suspensions, respectively, as well as with frits for separating the phases.

According to a particular embodiment, the kit is provided as a container which contains the ligands as well as the agents b) and c). In case of one single agent instead of two different agents b) and c), it may be advantageous to use this agent also together with the ligands in a formulation. Thus, e.g., a carrier of ligands may be selected which is also used for recovering antibodies and is effective as an adjuvant. Such a carrier is, e.g., a poorly soluble aluminum compound, such as AluGel or aluminum hydroxide. Even though in that case these ligands are contained in the pharmaceutical preparation together with the recovered antibodies, this is very much desired in case of an antibody ligand which itself is effective as a vaccination antigen.

The binding of antibodies from fluids from individuals to the ligands may be batch-wise or in the flow-through procedure. The immunoaffinity purification may occur automatically on a chromatography apparatus, or by means of a manual procedure; it is, however, also conceivable that the method is manually, automatically or semi-automatically carried out by means of a simple device which contains the immobilized ligands.

Finally, for the inventive isolation of antibodies from an individual, it is only necessary that the desired antibodies can substantially be separated from undesired other substances from the body. It is conceivable that this object can be achieved by separating methods other than immunoaffinity purification, as described above, such as, e.g., by reaction of ligands with the antibodies and a subsequent separation of the specific immune complexes from the substances which have not been complexed with the ligands. The antibodies may also be bound to the ligands, and recovered, respectively, in the liquid phase, in a colloidal solution, emulsion, or by the so-called immune-affinity partitioning.

Accordingly, the present invention also relates to a method of producing an autologous-antibody-containing vaccine, characterized by the following steps:

providing an antibody-containing fluid from an individual,

treating the fluid with ligands which bond to a certain group of the antibodies, with the proviso that as said ligands no antibodies or their fragments with the same idiotype are used which are directed against tumor-associated antigens,

separating all substances which do not bond to the ligands,

treating the antibodies bound to the ligands with an eluting agent so that the antibodies bound to the ligands are eluted,

recovering the antibodies which bond to the ligands in an eluate, and

working up the eluate obtained into an autologous vaccine that contains an immunogenic amount of more than one microgram of antibodies.

By the vaccine according to the invention, undesired antibody activities are substantially down-regulated. Inhibiting antibodies are, e.g., a target and, according to the invention, they can be isolated and formulated to a vaccine against these undesired inhibiting antibodies. The vaccine produced according to the invention is substantially utilized for the prophylactic and/or therapeutic application in syndromes which are connected to tumor diseases, autoimmune diseases, allergies or infectious diseases. Undesired immune reactions which may occur in the course of transplantations are a further field of indication.

Thus, e.g., patients can be treated who generate antibodies to sperms and thus exhibit an acquired sterility. If these autologous antibodies are removed from a body fluid, such as vaginal secretion, and employed according to the invention for producing a vaccine, this vaccine can be used immediately to treat the female patient so as to suppress the undesired antibodies. The vaccine preparation obtained will particularly contain IgA antibodies which again can be taken up primarily via the mucosa. The preferred delivery therefore is by nasal or vaginal administration, respectively.

With the assistance of an inventively produced antibody vaccine, also the rhesus factor incompatibility reaction of female patients can be treated. Women who are Rh-negative and have been contacted with blood or tissue, respectively, from Rh-positive persons develop antibodies against this rhesus factor. Thus, e.g., an Rh-negative female patient who is pregnant with an Rh-positive fetus may develop antibodies against the rhesus factor. These must be suppressed so as to avoid incompatibility reactions during a second pregnancy with an Rh-positive child. Therefore, antibodies against the rhesus factor are recovered according to the invention, e.g. from serum, and formulated to an immunogenic vaccine. The active immunization preferably is effected as a prophylaxis before a planned pregnancy.

The transplantation of allogenic material, such as bone marrow or stem cells, can also be assisted by a vaccine produced according to the invention. Possible rejection reactions by HLA antigen structures can be suppressed by administering a vaccine which contains the autologous antibodies against the foreign HLA.

Preparing xenotransplantations is a further field of application so as to prevent possible incompatibility reactions due to the transplant. High titers of natural antibodies against the known α-Gal-epitope, as they are found in humans in serum, are co-responsible for acute rejection reactions against xenotransplants or allogenic transplants. These natural anti-α-Gal antibodies are formulated into a vaccine with the help of the method according to the invention, and administered to the patient who is being prepared for the transplantation of tissue, bone marrow or stem cells. The down-regulation of the anti-α-Gal activity is to help avoid the rejection reactions. Lowering the titer of the circulating anti-α-Gal antibodies by immunization with autologous anti-α-Gal antibodies should make it possible to significantly increase the survival time of xenotransplants.

The choice of the ligands for the inventive isolation of antibodies will depend on the respective use thereof. In the following, a few applications are mentioned by way of example:

Application in autoimmune diseases:

Auto-antigens as ligands may, e.g., be used for isolating autoimmune-specific antibodies from an individual. After having been used according to the invention, antibodies recovered in this manner can elicit an immune response in an individual which causes a particular down-regulation of the production of the specific auto-antibodies by idiotypic interactions.

In many autoimmune diseases, the precise specificity of the autoreactive antibodies is not known. However, it is not absolutely necessary that autoantigens are used as a ligand for the isolation of autoimmune-specific antibodies according to the present invention. In case of an autoimmune disease, an antibody specificity may be present in an extremely over-proportional amount so that by purifying the entire immunoglobulin fraction (according to known biochemical methods) from an individual and subsequently formulating it as a vaccine and applying it to the donor individual thereafter, primarily anti-idiotypic antibodies are elicited against the over-proportionally represented antibody specificity.

By way of example, patients suffering from disorders associated with inhibiting antibodies against coagulation factors, insulin or also rheumatoid factors, are treated with a vaccine produced according to the invention. The vaccines utilized therefor contain the antibodies against the inhibiting antibodies or the rheumatoid factors.

Application in allergies:

To prepare a patient-specific antibody vaccine against allergies, e.g. anti-IgE antibodies or parts of such antibodies with the same specificity are, e.g., conceivable as ligand. Yet also allergens or parts of allergens are conceivable as ligand.

Application with toxic substances:

To purify from an individual antibodies which can trigger a toxin-specific immune response, toxin-specific antibodies can be used as ligands. In many cases, such antibodies are available as monoclonal antibodies. It is, however, also conceivable that not antibodies, but other molecules which are capable of binding quite specifically certain toxins (e.g. bacterial toxins or low-molecular toxins), are used as ligand for purification purposes.

Instead of only one ligand species, of course, also two or more ligands of different specificities can be immobilized on the solid carrier by means of the present method, and in this manner, antibodies can be obtained in preparations with specificities which are enriched or depleted, respectively, in terms of several binding properties (antibodies with different specificities). Analogously, also the arrangement in series of several immune adsorption steps with different specificities each is preferred in the preparation of multi-specific vaccines according to the present invention.

It is also possible to provide a solid carrier with several different ligands which are all directed to the same or a similar target substance (a polyclonal antibody mixture against a certain antibody class in the simplest case). Just as well, however, also e.g. different monoclonal antibodies against one and the same target structure can be provided on the solid carrier.

Particularly preferred ligands according to the present invention are autoantigens, such as, e.g., double-stranded DNA, so as to treat patient-specific antibodies against ds-DNA from patients with systemic Lupus erythematosus (SLE). Factor VIII or parts thereof can be used as ligand so as to isolate highly factor VIII-binding antibodies from an individual and to down-regulate the pathogenic anti-factor VIII reactivity of a patient with the vaccine formulated therefrom (Semin. Thromb. Hemost. (2000) 26:151). From patients afflicted with Myasthenia gravis, individual antibodies can be purified by immunoaffinity purification with acetylcholine receptor or parts thereof (Proc. Natl. Acad. Sci. USA (1993) 90:8747), which, formulated according to the invention as an autologous vaccine, can again be re-vaccinated into the same patients.

Insulin as a ligand can be used in the preparation of an autologous vaccine against autoimmune diabetes (type I insulin-dependent diabetes mellitus) (Diabets Metab. Res. Rev. (2000): 16:338).

Myelin basic protein (MBP) or parts thereof can be used as a ligand in the preparation of an autologous vaccine for multiple sclerosis patients or patients with other immunologically-caused neurological disorders (J. Neuroimmunol. (2001) 113:163).

Various gangliosides can be used as ligand (in patients with Guillain-Barre syndrome (Intern. Med. (1997) 36:599) and other neuropathies.

The advantage of this autologous type of immunization resides in the inherent consideration of the patient-specific idiotypes.

A further preferred ligand according to the present invention is an anti-human IgE antibody with which highly specific IgE fractions can be purified and which again can be administered to the patient in a suitable, immunogenic form so as to inhibit specific IgE-producing cells in patients. Of course, parts of specific anti-IgE antibodies, if they still have the desired specificity, can also be used as ligand.

In the field of allergy, it is conceivable to use as the ligand highly specific allergens or parts thereof or anti-idiotypic antibodies, or other molecules which mimic allergens.

Since the immune response induced by vaccination with autologous antibodies will be determined by the binding region of these antibodies, i.e. by their idiotype, in principle also fragments or derivatives of these antibodies may be used instead of intact antibody fractions for immunizing purposes, as long as they contain the idiotype of the respective starting antibody. The term “antibody” thus also comprises fragments or derivatives of such antibodies having the same binding specificity. As examples, yet without being restricted thereto, the following shall be mentioned: F(ab)2′ fragments, F(ab)′ fragments, which may, e.g., be produced according to biochemical methods known per se (e.g. by enzymatic cleavage). The term “derivative” comprises e.g. antibody derivatives which may be produced according to chemical or biochemical methods known per se, such as, e.g., with antibodies amidated with fatty acids at free amino functions for the purpose of increasing the lipophiles for incorporation in liposomes. In particular, the term also encompasses products which can be produced by chemical coupling of antibodies or antibody fragments with molecules which are capable of enhancing the immune response, such as, e.g., tetanus toxoid, Pseudomonas exotoxin, derivatives of lipid A, GM-CSF, IL-2, IL-12, C3d.

The shift of the immunological balance caused by a first vaccination can be further increased by repeating this procedure, e.g. a few weeks after recovering the first autologous vaccine by immunoaffinity purification, body fluid, e.g. blood, may again be taken, and again an autologous vaccine may be produced and administered. In this way it is also ensured that the respective status of the immunological balance will always be taken into consideration in the individual vaccine. This procedure can be repeatedly carried out at suitable intervals (e.g. every 4-8 weeks at first, and every 6 months later on), in accordance with a progress control of the immune status of the respective patient by a corresponding specific testing. The here described new composition and method of vaccinating with autologous antibodies is basically suitable both for therapeutic and also for prophylactic purposes.

One general advantage of the strategy of the individual autologous vaccination described here resides in the fact that the immunological status of the respective individual regarding the idiotypic network is taken into consideration, since the respective vaccine in each case is prepared from the individual body fluid, e.g. serum. Furthermore, the immunized individual does not get into contact with any foreign antigens, but is treated in suitable form with endogenous components only, which cause a modulation of the immunological balance.

One special application resides in the treatment of patients who have formed specific antibodies due to an immunization. The so-called hyperimmune serum is then used for producing an autologous vaccine so as to provoke an immune response against the autologous antibodies in due time.

In a preferred embodiment, according to the invention the antibodies obtained by immunoaffinity purification are formulated with a suitable vaccine adjuvant.

As is common in vaccines, the autologous antibody fractions or the fragments and derivatives thereof can be formulated together with vaccine adjuvants. By such adjuvants, the immune response is enhanced. As examples of adjuvants, yet, without being restricted thereto, the following shall be mentioned: aluminum-containing adjuvants, in particular aluminum hydroxide (e.g. AluGel), derivatives of lipopolysaccharide, Bacillus Calmette Guerin (BCG), saponines and derivatives thereof (e.g. QS-21), liposome preparations, formulations with additional antigens against which the immune system has already produced a strong immune response, such as, e.g. tetanus toxoid or components of influenza viruses, optionally in a liposome preparation.

To enhance the immune response, the vaccine preparation may also be administered with appropriate, preferably human, cytokines which assist in the buildup of an immune response. Here, in particular, though not exclusively, granulocyte macrophage-stimulating factor (GM-CSF) should be mentioned. This cytokine stimulates an efficient immune response by activating antigen-processing cells (e.g. dendritic cells).

Optionally, the autologous antibody fractions can also be incubated, according to per se known and published methods, with autologous, ex vivo cultured dendritic cells. The thus pulsed dendritic cells subsequently are administered again to the respective individual. In this manner, a particularly efficient immune response can be achieved.

Accordingly, in a preferred method according to the present invention, the working up of the antibody eluates includes the addition of a substance selected from the group of adjuvants, in particular aluminum-containing adjuvants, lipopolysaccharide derivatives, Bacillus Calmette Guerin, liposomes or QS-21 (further preferred adjuvants are described i.a. in Singh et al., Nat. Biotechnol. 17 (1999), pp. 1075-1081), immunostimulating cells, in particular dendritic cells or other antigen-presenting cells, active agents, preferably cytokines, in particular granulocyte macrophage-stimulating factor, formulating auxiliaries, in particular buffer substances, stabilizers or solubilizers, or mixtures of these substances.

In a preferred embodiment of the method according to the invention, the antibodies contained in the composition are mixed with an adjuvant and subesquently are subjected to a heat treatment, preferably at a temperatur of more than 80° C., in particular of between 90° C. and 130° C. The adjuvant used preferably is an aluminum-containing adjuvant. It is possible that such a heat treatment does denature the protein antigen, yet that the immunogenic portions of the protein, by binding to the adjuvant, can be presented to the immune system in the correct form. Yet, it is not absolutely necessary to denature the proteins so as to obtain the advantages of a heat treatment. It has been known that the thermal denaturing of proteins does not only depend on the temperature, but also on the time for which the protein is subjected to this temperature. Moreover, also further physical-chemical parameters, such as, e.g., ionic strength, ionic composition, pH, type and amount of the active surface in the mixture, are responsible for the denaturing of a protein. Conditions under which the antibodies are not, or not completely, denatured and/or other effects can be utilized, such as, e.g., a slighter desorption from the surface of the adjuvant, are known and can easily be optimized for any eluates by the person skilled in the art.

A further advantage of such a mode of producing a vaccine formulation with an adjuvant and the subsequent heat treatment is that infectious pathogens in the entire formulation could be attenuated or inactivated, respectively. This advantage may play a role both in the production and also in the storage and distribution of the vaccine formulation. With this, a higher safety with respect to known and unknown pathogens of communicable diseases is given. Moreover, with an appropriate packing, a filling into containers without preservatives is possible, since the microbial preservation of the vaccine has been effected by heat.

A further advantage of such a formulation is the possible increased immunogenicity of the antibodies, since heating may cause at least a partial denaturing of the antibodies. This increased antigenicity may increase the immunogenicity particularly in proteins which would be recognized by the immune system as endogenous proteins.

A further advantage resides in the additional stabilizing of the antibody-adjuvant complex by the thermal inactivation, i.e. the desorption of the protein-antigen is no longer rapid as in antigen-adjuvant formulations which have not been heat-treated. This advantage also allows for a longer time interval between the individual immunizations.

Accordingly, a particular embodiment of the method according to the invention relates to a method in which a protein-denaturing step, in particular a heat treatment, is effected in which the proteins contained in the eluates are at least partially changed in their three-dimensional structure, their immunogenic properties preferably being enhanced.

The composition produced according to the invention may be administered according to conventional methods, e.g. as a vaccine by subcutaneous, intramuscular or intradermal injection. A further mode of administration is via the mucosal pathway, e.g. the vaccination by nasal or peroral administration.

The present invention also relates to pharmaceutical compositions containing antibodies recovered by immunoaffinity purification from animal body fluids that contain antibodies, to be used as autologous vaccines.

Furthermore, the present invention relates to a method for the therapeutic or prophylactic vaccination against autoimmune diseases, infectious diseases, various intoxications and allergies.

Accordingly, the present invention also relates to an autologous vaccine obtainable according to the method of the invention.

An object of the present invention is also a method of treating individuals, wherein an inventively produced preparation is administered in an efficient amount, preferably a few micrograms to up to 10 grams to an individual from whom the body fluid has been taken. The efficiency must primarily be evaluated in terms of the immunogenicity. It has proven successful to use at least one microgram of antibody in a vaccine dose which can be administered in ready-to-use dose units of from 0.01 to 1 ml, preferably in the range of from 0.1 to 0.5 ml. The preferred amount will primarily depend on the supporting effect of adjuvants and is in the range of from 3 micrograms to 1 gram, particularly preferred 10 micrograms to 750 micrograms, most preferred 250 micrograms to 500 micrograms.

Primarily also in autoimmune diseases, such as, e.g., systemic Lupus erythematosus, autoimmune thyroiditis, systemic vasculitis, Guillain-Barre syndrome and anti-factor VII:C autoimmune disease, in allergies, in tumor diseases and in the prophylaxis of incompatibility reactions within the scope of transplantations as well as in intoxications (such as, e.g., with bacterial toxins), this treatment method is particularly effective.

According to a further aspect, the present invention also relates to the use of an autologous antibody preparation for producing a means for immunomodulation.

The invention will be explained in more detail by way of the following examples and the drawing figures to which, however, it shall not be restricted.

Therein,

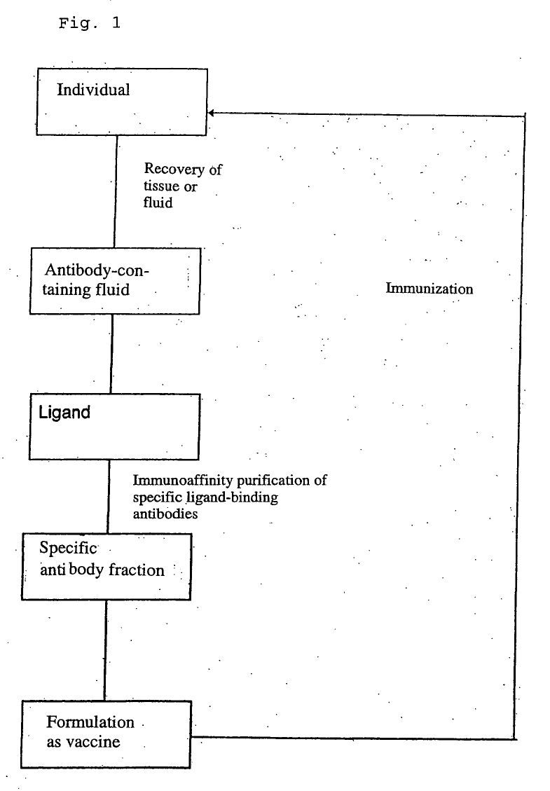

FIG. 1 shows a diagram of the recovery of the vaccine;

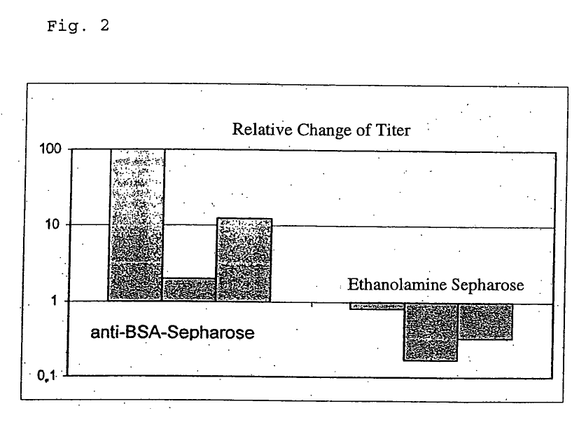

FIG. 2 shows the change of the specific antibody reactivities after immunization with autologous vaccine, prepared via anti-bovine serum albumin as ligand, or Sepharose, respectively;

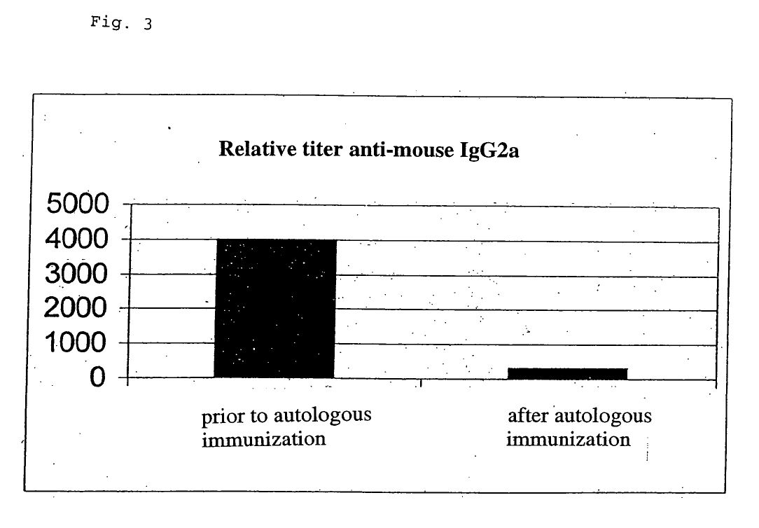

FIG. 3 shows the change of the specific antibody reactivity after immunization with an autologous vaccine, prepared via mouse-IgG2a as ligand.

FIG. 4 shows a system of vessels for producing the autologous vaccine, using magnetic particles.

### EXAMPLES

### Example 1

This example is intended to illustrate that it is possible to specifically modulate the immune system against any desired protein (bovine serum albumin, BSA, in this instance).

Two groups of 3 rabbits each were immunized with a vaccine formulation produced according to the invention.

The autologous vaccine for the first group was produced by purifying immunoglobulin from the serum of the rabbits via an affinity chromatography column (rabbit anti-BSA immobilized on Sepharose). The autologous vaccine for the second group was produced by purifying immunoglobulin from the serum of rabbits via a different affinity-chromatographic column (Sepharose without specific ligands).

The immunoglobulins thus obtained were formulated as a vaccine by adsorption on aluminum hydroxide gel and administered subcutaneously to the respective rabbits.

Blood drawing and vaccination regimen:

Day −21: recovery of serum

Day −14: recovery of serum

Day −7: recovery of serum

From the pool of the sera of days −21, −14 and −7, the vaccine was purified.

Day 0: recovery of serum

Day 0: immunization, subcutaneous

Day 14: recovery of serum

Day 21: recovery of serum

Preparation of the affinity matrix:

In a first step, the. anti-BSA serum of rabbits was purified:

For this purpose, the polyclonal rabbit-anti-BSA-antibodies (in 0.1 M glycine/HCl buffer, pH 2.9; volume=4 ml) were dialysed against dialysis buffer (0.1 M NaHCO₃+0.5 M NaCl pH=8.0) (Slide-A-Lyzer\grave{O}10 K; Pierce/USA).

Method:

It was dialyzed in a 800 ml beaker glass with magnetic stirring rod on the magnetic stirrer at 4° C., with the dialysis buffer being renewed four times.

Then the samples were concentrated by centrifuging (with Centricon 10 K (Amicon)), final volume: 0.4-0.6 ml.

Immobilization on activated CH-Sepharose:

Materials:

Activated CH-Sepharose 4B; Pharmacia Biotech (Code No. 17-0490-01)

Ligand: polyclonal anti-BSA-antibody (6.6 mg/ml, in coupling buffer)

Coupling buffer: 0.1 M NaHCO₃+0.5 M NaCl, pH=8.0

1 mM HCl

1 M Ethanolamine solution

0.1 M Tris-HCl (Tris(hydroxymethyl)-aminomethane) buffer, pH=8.0

0.1 M Tris-HCl buffer+0.5 M NaCl, pH=8.0

0.1 M Acetate buffer+0.5 M NaCl, pH=4.0

Coupling method:

0.25 g of freeze-dried CH-Sepharose 4B were suspended in approximately 20 ml of 1 mM HCl, washed with 50 ml of 1 mM HC1, subsequently washed with 50 ml of coupling buffer. The Sepharose was transferred into a 50 ml Falcon tube, and the antibody solution was added. A ratio of gel:buffer=1:2 results in an adequate suspension for coupling. The suspension was shaken for approximately 1 h. Excessive antibody was removed by washing with 3×10 ml of coupling buffer. Remaining active binding sites were blocked by means of a one-hour incubation on the shaker with 1 M ethanolamine. There followed a one-hour incubation of the Sepharose with 0.1 M Tris-HCl buffer (pH=8.0) on the shaker and the following washing cycle: at first, washing with 0.1 M acetate buffer (pH=4.0)+0.5 M NaCl, then with 0.1 M Tris-HCl buffer (pH=8.0)+0.5 M NaCl. The washing cycle was carried out three times.

Preparation of the affinity matrix for the second group of rabbits:

The affinity matrix for the second group (control group) was produced according to the same procedure as described above. Instead of the antibody solution, only buffer was used. Activated Sepharose thus is exclusively blocked with ethanolamine:

Production of the autologous vaccines:

10 ml serum each of the respective rabbits (pool of days −21, −14 and −7) were purified with the respective affinity matrices (0.5 ml column volume).

Materials:

Application buffer: PBS+0.2 M NaCl, pH=7.2

Elution buffer: 0.1 M glycine/HCl buffer, pH=2.0

Method:

Application on the column was with an incubation time of 30 min at +4° C. Washing was effected with application buffer (1 washing step=5 ml; 5 washing steps). Elution was effected with 1 ml of elution buffer.

The eluate was neutralized with carbonate solution (0.5 M NaHCO₃), the thus purified proteins were analyzed by means of size exclusion chromatography.

Size exclusion chromatography:

The chromatography was performed with a ZORBAX GF-250 column on a DIONEX-HPLC system. As the quantitative standards, the following immunoglobulins were used:

human IgG standard (20 mg/ml; Sandoglobulin, 3.590.009.0)

human IgM standard (1.1 mg/ml; SIGMA, cat.I-8260)

Column: ZORBAX GF 250 (PN: 884973.901)

Running buffer: 220 mMol NaPO₄ buffer, pH 7.0+10% acetonitrile

Flow rate: 1,000 ml/min

Wave length: 214 nm

Band width: 5 nm

Injection volume: 50 μl

Formulation with aluminum hydroxide:

The formulation of the purified, neutralized immunoglobulins as vaccine was carried out according to the following procedure:

For each vaccine, a Centricon ultrafiltration unit (Centricon 10 K from Amicon, USA) was used. At first, the ultrafiltration unit was washed (by centrifuging of 1 mM Na phosphate buffer, 0.86% NaCl, pH 6 (NBK). Subsequently, 400 μl of buffer and alhydrogel (27 μl (for 400 μl), Superfos, Denmark) were charged, the neutralized eluate was added, centrifuged and washed (with 5 ml of buffer) so that the final volume was approximately 300 μl. This was followed by re-suspension of the vaccines and the filling up with buffer to 396 μl, the addition of 4 μl of thimerosal stock solution (10 mg/ml, Sigma), 350 μl thereof were sterile-filled into containers. The protein concentrations of the individual vaccines was 40-50 μg each.

BSA-ELISA:

The following samples were analyzed in the ELISA: day 0, as well as a pool of day 14 and day 21. ELISA-plates (NUNC, Maxisorp (F=96); Denmark) were coated with 100 μl of BSA solution (per well). (BSA solution: BSA (SIGMA cat. No. A-7638); 10 μg/ml in coating buffer). It was incubated for 1 h at 370C. After washing, it was blocked with 5% of dry milk in PBS (200 μl/well). Incubation: 30 min at 37° C.

Washing Regimen:

After coating, blocking and sample incubation: 6 times with washing buffer, after the 4^{th} time, incubating for one minute.

after conjugate: 4 times with washing buffer, twice with staining buffer.

Serum samples were serially diluted (in 2% dry milk/PBS). The sample dilutions (100 μl/well) were incubated for 1 h at 37° C. As the positive control and as standard for a quantitative evaluation of the ELISA, a dilution series of the polyclonal rabbit-anti-BSA serum was used which had been used for the affinity purification. After washing, the enzyme conjugate (anti-rabbit Ig HRP (Nordic Immunology, #4694)) was applied in the appropriate dilution (1:1000 dilution buffer) (100 μl/well). After an incubation of 30 min at 37° C., it was washed again, substrate was added (per well: 100 μl of TMB Microwelll (BioFX, cat-No:TMBW-0100-01, #0034302)), and after appropriate staining, the reaction was stopped (with 30% H₂SO₄ 50 μl/well), then the staining was measured in the photometer (450 nm). The titer was determined at the half-maximum adsorption of each dilution series. The relative change of titer to the zero serum (day 0;=time of immunization) for the individual rabbits is illustrated in FIG. 2. It can be recognized that the rabbits which had received an autologous vaccine which had been prepared by means of an anti-BSA affinity chromatography, exhibit a marked titer shift in the BSA-ELISA.

### Example 2

This example shall demonstrate that it is possible to specifically lower an already existing immune response in an individual. For this purpose, a rhesus monkey was used, which had been immunized with a monoclonal antibody (HE2, mouse-IgG2a) and had developed a strong IgG immune response against mouse-IgG2a. The serum of this monkey was purified on an immunoaffinity column on which the mouse-IgG2a (HE2) was immobilized as ligand. The immunoglobulins purified in this manner were formulated as a vaccine on aluminum hydroxide and inocculated to the donor monkey subcutaneously. At the time of immunization (prior to vaccination) as well as 2 weeks thereafter, blood was drawn so as to determine the specific immune response against mouse-IgG2a.

Material and methods:

Microtiter plates for ELISA: Immuno Plate F96 MaxiSorp

(Nunc)

| | |
|---|---|
| Coupling buffer: | 0.1 NaHCO₃ |
| | 0.5 M NaCl |
| | pH 8.0 |
| Purifying buffer A: | PBS + 0.2 M NaCl |
| Purifying buffer B: | 0.1 M glycine / HCl |
| | 0.2 N NaCl |
| | pH 2.9 |
| Binding buffer: | 15 mN Na₂CO₃ |
| | 35 mM NaHCO₃ |
| | 3 mN NaN₃ |
| | pH: 9.6 |
| PBS | 138 mM NaCl |
| | 1.5 mM KH₂PO₄ |
| | 2.7 mM KCl |
| | 6.5 mM Na₂HPO₄ |
| | pH: 7.2 |
| Washing buffer A: | 2% NaCl |
| | 0.2% Triton X-100 |
| | in PBS |
| Washing buffer B: | 0.05% Ween 20 in PBS |
| Blocking buffer A: | 5% fetal calf serum (heat-in- |
| | activated) in PBS |
| Blocking buffer B: | 1% bovine serum albumin |
| | 0.1% NaN₃ |
| | in PBS |
| Diluting buffer A: | 2% fetal calf serum (heat-in- |
| | activated) |
| | in PBS |
| Diluting buffer B: | PBS |
| Staining buffer: | 24.3 mM citric acid |
| | 51.4 mM Na₂HPO₄ |
| | pH: 5.0 |
| Substrate: | 40 mg o-phenylenediamine-dihydrochloride |
| | 100 ml of staining buffer |
| | 20 μl H₂O₂ (30%) |
| Stop solution: | 4 N H₂SO₄ |
| Formulating buffer: | 10% PBS, pH = 5.5 |
| | 90% physiological NaCl solution |

Implementation:

The method described here for the autologous vaccination was tested on a rhesus monkey which gave a strong immune response against mouse-IgG2a (0.5 mg mouse-IgG2a (HE2), absorbed on 1.67 mg of aluminum hydroxide in 0.5 ml 1 mM phosphate-buffer, pH 6.0/155 mM NaCl was vaccinated each on days 1, 15, 29 and 57 subcutaneously to a rhesus monkey). The sera of different points of time were tested for mouse-IgG2a-specific antibodies by means of ELISA (see below). The antibodies at the end of the vaccination regimen were primarily of the IgG type. From this rhesus monkey, 10 ml of peripheral blood were taken and serum was recovered therefrom. For the immunoaffinity purification of the antibody fraction from the serum of this rhesus monkey, at first an immunoaffinity matrix was prepared according to the following protocol.

The entire procedure was carried out under sterile conditions: 7.5 g of CH-Sepharose 4B (Pharmacia) were suspended for 15 min in 20 ml of 1 mM HCl. The gel was then washed with 1 liter of 1 mM HCl, and subsequently with 200 ml of coupling buffer on a sinter glass filter AG3. 100 mg of murine antibody HE2 (mouse-IgG2a) were dialyzed against 5 liters of coupling buffer and adjusted with coupling buffer to 5 mg/ml. This solution was mixed with the gel suspension in a closed vessel. A ratio of gel:buffer of 1:2 gives a suspension suitable for coupling. This suspension was rotated at 4° C. for 24 h. Subsequently, the excess of ligand was washed off with 3×30 ml of coupling buffer. Residual reactive groups were blocked by a 1 h incubation at 4° C. with 1 M ethanolamine. Then the gel was rotated for 1 h at room temperature with a 0.1 M Tris-HCl buffer. Finally, the gel was washed with 3 cycles of buffers with alternating pH. Each cycle consisted of 0.1 M sodium-acetate buffer, pH 4, with 0.5 M NaCl, and subsequently 0.1 M Tris-HCl buffer, pH 8, with 0.5 M NaCl. The gel was stored at 4° C. The immunoaffinity purification of the antibody fraction from the serum of a rhesus monkey was effected according to the following protocol under sterile conditions: the immunoaffinity purification was carried out on an FPLC system (Pharmacia). 1 ml of the gel obtained according to the above protocol was filled into a Pharmacia HR5/5 column. 5 ml of serum were diluted 1:10 with the purifying buffer A. This solution was pumped over the column at 1 ml/min, and it was further washed with purifying buffer A, until the UV base line of the detector is reached again (280 nm). Bound immunoglobulins were then eluted with purifying buffer B, and the fraction was neutralized with 1 M Na₂HPO₄ immediately after desorption. 50 μl of the thus purified antibody fraction were analyzed on a size exclusion column (SEC, Zorbax 250 GF). As the running agent, 220 mM phosphate buffer, pH 7+10% acetonitrile was used. The entire amount of the antibody fraction was approximately 40 μg (determined by means of SEC as compared to a standard). The thus recovered antibody fraction was tested in an ELISA regarding the binding to antibody HE2 (which was used as ligand for the affinity purification): 100 μl aliquots of the mouse-IgG2a antibody used for the affinity purification (antibody HE2; solution with 10 μg/ml in binding buffer) were incubated in the wells of a microtiter plate for 1 h at 37° C. After having washed the plate six times with washing buffer A, 200 μl each of the blocking buffer A were added and it was incubated for 30 min at 37° C. After having washed the plate as described above, 100 μl aliquots each of the affinity-purified antibody fraction to be tested as well as normal human immunoglobulin at the same concentration as negative control in dilutions of 1:4 to 1:65 000 in diluting buffer A were incubated for 1 h at 37° C. After having washed the plate as described above, 100 μl each of the peroxidase conjugated goat-anti-human-Ig antibody (Zymed) in a dilution of 1:1000 in diluting buffer A were added and incubated for 30 min at 37° C. The plate was washed four times with washing buffer A and twice with staining buffer. The antibody binding was proven by the addition of 100 μl each of the specific substrate, and the color reaction was stopped after approximately 3 minutes by adding 50 μl each of stop solution. The evaluation was effected by measuring the optical density (OD) at 490 nm (wave length of the reference measurement is 620 nm). The affinity-purified antibody fraction showed a marked binding to the mouse-IgG2a antibody, whereas normal human immunoglobulin practically does not bind.

The antibody fraction obtained by affinity purification was formulated with aluminum hydroxide as adjuvant according to the following protocol:

3 ml of the antibody solution obtained after affinity chromatography (containing approximately 40 μg of antibody) were admixed with 1 mg of aluminum hydroxide (aqueous suspension; Alhydrogel, Superfos), and the suspension was centrifuged in a “FILTRON” centrifuge tube (Microsep TM, cut-off 10KD) at 4000×g for 30 min. Subsequently, it was slurried 2× with 1 ml each of the formulating buffer and centrifuged for 30 min at 4000×g. The suspension was filled up with formulating buffer to 0.5 ml, and the thus obtained suspension was sterile-filled into containers. The rhesus monkey of whose serum the above autologous vaccine was recovered was subcutaneously vaccinated into the back with this vaccine. Before this first vaccination, 5 ml of blood were drawn for serum recovery (to determine the starting value for characterizing the immune response). Two weeks later, again 10 ml of blood were drawn for serum recovery.

The binding of the serum immunoglobulin of this immunized monkey to mouse IgG2a was determined in the ELISA as above. As can be seen in FIG. 3, after the immunization with the autologous vaccine, the mouse-IgG2a reactivity drops.

1. A method of producing an autologous-antibody-containing vaccine, which method is characterized by the following steps:
providing an antibody-containing fluid from an autologous-antibody-containing human body fluid or from autologous cell or tissue preparations,
treating the antibody-containing fluid with a solid carrier on which ligands have been immobilized which bond to a certain group of antibodies, with the proviso that, as said ligands, no antibodies or their fragments with the same idiotype are used which are directed against tumor-associated antigens,
recovering the antibodies which bond to the ligands, and
working up the recovered antibodies to an autologous vaccine which comprises an immunogenic amount of more than one microgram of antibodies, by adding a substance selected from the group of adjuvants.

2. A method of producing an autologous-antibody-containing vaccine, which method is characterized by the following steps:
providing an antibody-containing fluid from a human individual,
treating the fluid with ligands which bond to a certain group of the antibodies, with the proviso-that, as said ligands, no antibodies or their fragments with the same idiotype are used which are directed against tumor-associated antigens,
separating all substances which do not bond to the ligands,
treating the antibodies bound to the ligands with an eluting agent so that the antibodies bound to the ligands are eluted,
recovering the antibodies which bond to the ligands in an eluate, and
working up the eluate obtained into an autologous vaccine that contains an immunogenic amount of more than one microgram of antibodies, by adding a substance selected from the group of adjuvants.

3. A method according to any one of claims 1 or 2, characterized in that an autologous vaccine containing an immunogenic amount of antibodies in the range of from 3 micrograms to 3 grams is produced.

4. A method according to any one of claims 1 to 3, characterized in that a means for binding antibodies which occur in autoimmune diseases is chosen as ligands.

5. A method according to any one of claims 1 to 3, characterized in that a means for binding antibodies which occur in allergic diseases is chosen as ligands.

6. A method according to any one of claims 1 to 3, characterized in that a means for binding antibodies which are directed against the α-Gal-epitope are chosen as ligands.

7. A method according to any one of claims 1 to 3, characterized in that a means for binding antibodies which are directed against human sperms is chosen as ligands.

8. A method according to any one of claims 1 to 3, characterized in that a means for binding antibodies which are specific for B-cell lymphoma diseases is chosen as ligands.

9. A method according to any one of claims 1 to 8, characterized in that the body fluid is serum or plasma.

10. A method according to any one of claims 1 to 9, characterized in that the individual is a human being.

11. A method according to any one of claims 1 to 10, characterized in that the ligands are chosen from more than one type, in particular ligands which bind antibodies with different specificity.

12. A method according to any one of claims 1 to 11, characterized in that the ligands bind a certain subclass of immunoglobulins.

13. A method according to any one of claims 1 to 12, characterized in that the ligands bind certain immunoglobulin chains.

14. A method according to any one of claims 1 to 13, characterized in that antibodies, in particular monoclonal antibodies, antigens, autoantigens, haptens, allergens or mixtures thereof are used as ligands.

15. A method according to any one of claims 1 to 14, characterized in that the working up comprises the addition of a substance selected from the group of adjuvants, in particular aluminum-containing adjuvants, lipopolysaccharide derivatives, Bacillus Calmette Guerin, liposomes, saponines and derivatives thereof, immuno-stimulating cells, in particular dendritic cells, active agents, preferably cytokines, in particular granulocyte macrophage stimulating factor, formulating auxiliaries, in particular buffer substances, stabilizers or solubilizers, or mixtures of these substances.

16. A method according to any one of claims 1 to 15, characterized in that a protein-denaturing step, in particular a heat treatment, is carried out.

17. An autologous vaccine, characterized in that it is obtainable according to a method as set forth in any one of claims 1 to 16.

18. The use of an autologous vaccine according to claim 17 for producing a means for immunomodulation, in particular for down-regulating undesired antibody activities.

19. A kit for producing an autologous vaccine, said kit comprising the components:
a) a device with ligands for binding a certain group of antibodies from an antibody-containing fluid of an individual,
b) an agent for recovering the antibodies which bond to the ligands, and
c) an agent for working up the recovered antibody to an autologous vaccine, said means comprising an adjuvant.

20. A kit according to claim 19, characterized in that the component a) comprises a container for receiving the ligands and the antibody-containing fluid.

21. A kit according to claim 20, characterized in that the container contains the ligands bound to a solid carrier.

22. A kit according to claim 21, characterized in that the container contains magnetic beads and that the kit further comprises a magnet for localizing the beads.

23. A kit according to any one of claims 19 to 22, characterized in that the component b) comprises a means for purifying the antibody.

24. A kit according to any one of claims 19 to 23, characterized in that a single agent for recovering and working up the recovered antibodies, and optional auxiliary agents are contained as component b) and c).

25. A kit according to claim 24, characterized in that the means contains a poorly soluble aluminum compound for recovering and working up the recovered antibodies.

26. A kit according to any one of claims 19 to 25, characterized in that the device a) comprises a container containing the ligands, the means b) and c).

27. A kit according to claim 26, characterized in that the device comprises a syringe containing an vaccine and an adjuvant.
